(19) United States
(12) Patent Application Publication
(10) Pub. No.: US 2024/0087752 A1
(43) Pub. Date: Mar. 14, 2024

(54) SYSTEMS AND METHODS FOR MULTI-LANGUAGE ADAPTIVE MENTAL HEALTH RISK ASSESSMENT FROM SPOKEN AND WRITTEN LANGUAGE

(71) Applicant: Ellipsis Health, Inc., San Francisco, CA (US)

(72) Inventors: Tomasz RUTOWSKI, San Francisco, CA (US); Amir Hosein HARATI NEJAD TORBATI, San Francisco, CA (US); Elizabeth E. SHRIBERG, San Francisco, CA (US)

(21) Appl. No.: 18/263,650

(22) PCT Filed: Feb. 3, 2022
(86) PCT No.: PCT/US2022/015147

§ 371 Date: Jul. 31, 2023

Related U.S. Application Data
(60) Provisional application No. 63/145,376, filed on Feb. 3, 2021.

(51) Int. Cl. G16H 50/30; G10L 15/00; G10L 15/18; G10L 25/66; G16H 15/00

(52) U.S. Cl. CPC G16H 50/30; G10L 15/005; G10L 15/18; G10L 25/66; G16H 15/00

(57) ABSTRACT

Disclosed is a method for detecting a behavioral or mental health condition. The method includes (a) receiving an input signal comprising a plurality of audio or lexical characteristics of speech of a subject. At least one of the plurality of audio or lexical characteristics of the speech relates to at least one language. The method also includes (b) based at least in part on the plurality of audio or lexical characteristics of the input signal, selecting one or more acoustic or natural language processing (NLP) models. At least one of the acoustic or NLP models is a multi-lingual or language-independent model. The method also includes (c) detecting a result indicating a presence or absence of the behavioral or mental health condition by processing the input signal with a joint model or a fused model derived from the one or more acoustic or natural language processing models.

### CROSS-REFERENCE

This application claims priority to U.S. Provisional Patent Application No. 63/145,476, filed on Feb. 3, 2021, which is entirely incorporated herein by reference.

### BACKGROUND

Behavioral and mental health conditions are prevalent in the population, and they may have a significant cost to society. Therapies are available for such conditions, but many people may be undiagnosed. Many behavioral and mental health screening systems that use acoustic and/or natural language processing modeling have been devised for speakers for which there is a large corpus of data available, such as English. But for languages that do not have large corpora of data, such analysis may be difficult or impossible.

### SUMMARY

The present disclosure provides systems and methods for predicting a risk severity of a subject for having behavioral and mental health conditions from speech in a multilingual scenario (for example, by using multilingual models, language-agnostic models, or language-independent models). The disclosed system may enable mental health risk prediction for input speech (in audio or text format) with content in unknown languages by using models that have been trained on or implemented using known languages.

The disclosed system may use acoustic and/or natural language processing models to perform the analysis. For example, acoustic models may be used to analyze audio data from speech, while NLP models may be used to analyze text and/or audio data. An acoustic model used by the system may adhere to or be based on one of many different architectures and should not be construed to being limited to a deep learning architecture where feature representations are learned from data. Acoustic models used herein may also be based on or include architectures where features are designed by experts. The system may adaptively choose between various acoustic and NLP models, or combinations therefrom, to make the predictions. The system may generate feedback based on the performance of the model, availability of data, topics, and configuration used.

The system may first retrieve a segment of speech data from a subject. The subject may be a human subject. The human subject may be a patient. The system may then detect one or more languages present in the speech. The system may, based on the one or more detected languages, estimate a model performance based on how well the models may perform on the language or languages. The system may, based at least in part on this estimate of model performance, select the combination of models to use with a pre-model strategy selector. The system may then implement a joint model, a weighted combination of the one or more models, an unweighted combination of the one or more models, a rule-based model, or another type of model to predict the condition. A post-model strategy selector may determine, based on a confidence value associated with the model's performance, whether an alternate form of analysis is necessary.

A feedback mechanism may change one or more configurations of the test or recommend one or more actions to the subject, agent, or application associated with the subject, agent, or another user.

The system may produce an electronic report using the risk severity it has determined for the behavioral or mental health condition. In some embodiments, the electronic report may comprise psychoeducation materials relating to the behavioral or mental health condition if the determination indicates that the subject has or is likely to have the behavioral or mental health condition.

In an aspect, a method for detecting a behavioral or mental health condition is disclosed. The method comprises (a) receiving an input signal comprising a plurality of audio or lexical characteristics of speech of a subject. At least one of the plurality of audio or lexical characteristics of the speech relates to at least one language. The method also comprises (b) based at least in part on the plurality of audio or lexical characteristics of the input signal, selecting one or more acoustic or natural language processing (NLP) models. At least one of the acoustic or NLP models is a multi-lingual or language-independent model. The method also comprises (c) detecting a result indicating a presence or absence of the behavioral or mental health condition by processing the input signal with a fused model or joint model derived from the one or more acoustic or NLP models.

n some embodiments, the input signal comprises text or audio.

n some embodiments, the at least one language is from a database of known languages.

In some embodiments, the at least one language has not been previously identified in a database of known languages.

In some embodiments, the method further comprises, prior to (b), determining a performance estimate for one or more acoustic or NLP models. The performance estimate is associated with one or more of the audio or lexical characteristics of the input signal.

In some embodiments, the performance estimate is determined using statistical modeling.

In some embodiments, processing the input signal with a fused model derived from the one or more acoustic or NLP models comprises (i) applying one or more weights to the one or more acoustic or NLP models; and (ii) combining the one or more weighted acoustic or NLP models.

In some embodiments, combining the one or more weighted acoustic or NLP models comprises concatenating one or more outputs of the one or more weighted acoustic or NLP models.

In some embodiments, the selecting the one or more acoustic or NLP models is also based at least in part on a latency of a prediction, a context of the input signal, or demographic information about the subject.

In some embodiments, the one or more NLP models include one or more of generic language models, translation-based models, or multilingual models.

In some embodiments, (c) further comprises fine-tuning at least one acoustic or NLP model.

In some embodiments, the behavioral or mental health condition includes depression, anxiety, stress, schizophrenia, bipolar disorder, or post-traumatic stress disorder (PTSD).

In some embodiments, the one or more acoustic models include one or more of acoustic multilingual models or acoustic language-independent models.

In some embodiments, an acoustic model of the one or more acoustic models is a supervised, semi-supervised, or unsupervised model.

In some embodiments, the joint model or fused model derived from the at least one or more acoustic or NLP models further comprises at least one demographic or metadata-based model.

In some embodiments, the method further comprises, in (b), determining whether to provide feedback to the subject.

In some embodiments, the feedback is modifying a question prompt to the subject, modifying a set of questions asked to the subject, eliciting more speech from the subject, asking a follow-up question to the subject, prompting the subject to take one or more actions to increase a signal-to-noise ratio for the input signal, or modifying a language of an interaction with the subject.

In an aspect, a method for detecting a behavioral or mental health condition is disclosed. The method comprises (a) receiving an input signal comprising a plurality of audio or lexical characteristics of speech of a subject. At least one of the plurality of audio or lexical characteristics of the speech relates to at least one language. The method also comprises (b) determining one or more certainty measures. A certainty measure of the one or more certainty measures corresponds to a language of the input signal. The method also comprises (c) determining at least one confidence measure based on at least one of the one or more certainty measures. A confidence measure is associated with a performance, with respect to the language of the at least one certainty measure, of an acoustic model or natural language processing (NLP) model of a plurality of acoustic or NLP models for detecting the behavioral or mental health condition. The method also comprises (d) based at least in part on the plurality of audio or lexical characteristics of the input signal and the at least one confidence measure, selecting one or more acoustic or natural language processing (NLP) models. At least one of the acoustic or NLP models is a multi-lingual or language-independent model. Finally, the method comprises (e) detecting a result indicating a presence or absence of the behavioral or mental health condition by processing the input signal with a fused model or joint model derived from the one or more acoustic or NLP models.

In an aspect, a method for detecting a behavioral or mental health condition is disclosed. The method comprises (a) receiving an input signal comprising a plurality of audio or lexical characteristics of speech of a subject. At least one of the plurality of audio or lexical characteristics of the speech relates to at least one language. The method also comprises (b) determining one or more certainty measures. A certainty measure of the one or more certainty measures corresponds to a language of the input signal. The method also comprises (c) determining at least one confidence measure based on at least one of the one or more certainty measures. A confidence measure is associated with a performance, with respect to the language of the at least one certainty measure, of an acoustic model or natural language processing (NLP) model of a plurality of acoustic or NLP models for detecting the behavioral or mental health condition. The method also comprises (d) based at least in part on the plurality of audio or lexical characteristics of the input signal and the at least one confidence measure, selecting one or more acoustic or natural language processing (NLP) models. At least one of the acoustic or NLP models is a multi-lingual or language-independent model. The method also comprises (e) detecting a result indicating a presence or absence of the behavioral or mental health condition by processing the input signal with a fused model or joint model derived from the one or more acoustic or NLP models. The method also comprises (f) based on a reliability measure of the result, iteratively selecting one or more remedial actions and repeating steps (d) and (e) until a reliability threshold is achieved.

In an aspect, a method for detecting a behavioral or mental health condition is disclosed. The method comprises (a) receiving an input signal comprising a plurality of audio or lexical characteristics of speech of a subject. At least one of the plurality of audio or lexical characteristics of the speech relates to at least one language. The method also comprises (b) determining one or more certainty measures. A certainty measure of the one or more certainty measures corresponds to a language of the input signal. The method also comprises (c) determining at least one confidence measure based on at least one of the one or more certainty measures. A confidence measure is associated with a performance, with respect to the language of the at least one certainty measure, of an acoustic model or natural language processing (NLP) model of a plurality of acoustic or NLP models for detecting the behavioral or mental health condition. The method also comprises (d) based at least in part on the plurality of audio or lexical characteristics of the input signal and the at least one confidence measure, selecting one or more acoustic or natural language processing (NLP) models. At least one of the acoustic or NLP models is a multi-lingual or language-independent model. The method also comprises (e) detecting a result indicating a presence or absence of the behavioral or mental health condition by processing the input signal with a fused model or joint model derived from the one or more acoustic or NLP models. The method also comprises (f) based at least in part on a reliability measure of the result, iteratively selecting one or more remedial actions and repeating steps (d) and (e) until a reliability threshold is achieved. The method also comprises (g) based at least in part on the result, determining one or more remedial actions to the subject or to an agent to improve collection of the input signal.

In some embodiments, the certainty measure is generated at least in part using metadata of the input signal.

In some embodiments, the method further comprises applying at least one label corresponding to at least one of the one or more languages of the input signal.

In some embodiments, the at least one confidence measure is based at least in part on a length of a session in which the input signal is recorded, a topic of the input signal, or a quality of the input signal.

In some embodiments, the method further comprises (i) determining whether the one or more acoustic or NLP models is unreliable; and (ii) if the one or more acoustic or NLP models is unreliable, determining one or more remedial actions to the subject or to an agent to improve collection of the input signal.

In some embodiments, a model of the one or more of acoustic or NLP models is a translation model.

In some embodiments, an acoustic model of the one or more of acoustic or NLP models is a multilingual acoustic model or a language-dependent acoustic model.

In some embodiments, the one or more acoustic or NLP models includes at least one metadata or demographic model.

In some embodiments, the one or more acoustic or NLP models comprises fusing outputs from at least two acoustic or NLP models.

In some embodiments, a result associated with the one or more remedial actions from (f) is provided in an electronic report.

In another aspect, the present disclosure provides a method for training a natural language processing model to detect a behavioral or mental health condition, wherein the natural language processing model comprises (i) a language model and (ii) a classifier, and wherein the method comprises: (a) training the language model on first encoded text, wherein the first encoded text comprises text that is unrelated to the behavioral or mental health condition; (b) fine-tuning the language model on second encoded text and optionally on metadata information, wherein the second encoded text comprises text that is related to the behavioral or mental health condition; and (c) training the classifier to detect the behavioral or mental condition on a plurality of encoded speech samples from a plurality of subjects, wherein an encoded speech sample of the plurality of encoded speech samples is associated with a label and optional metadata information that indicates whether a subject that provided the encoded speech sample has the behavioral or mental health condition.

In another aspect, a method for detecting a behavioral or mental health condition. The method comprises (a) computer processing an input signal comprising a plurality of audio or lexical characteristics of speech of a subject, wherein at least one of the plurality of audio or lexical characteristics of the speech relates to at least one language. The method also comprises (b) detecting a result indicative of a presence or absence of the behavioral or mental health condition using a model derived from one or more acoustic or natural language processing (NLP) models, wherein at least one of the acoustic or NLP models is a multi-lingual or language-independent model.

Another aspect of the present disclosure provides a non-transitory computer readable medium comprising machine executable code that, upon execution by one or more computer processors, implements the systems described above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

In another aspect, the present disclosure provides a system comprising: one or more computer processors; and memory comprising machine-executable instructions that, upon execution by the one or more computer processors, cause the one or more computer processors to implement an acoustic model configured to predict whether a subject has a behavioral or mental health state of interest based at least in part on input speech comprising a plurality of segments from the subject, As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates a system that is configured to predict whether a subject has a behavioral or mental health state of interest based at least in part on input speech from the subject, in accordance with an embodiment;

FIG. 2 schematically illustrates a system that is configured to assess, screen, predict, or monitor the behavioral or mental health state of a subject using speech data, video data, and/or metadata about the subject, in accordance with an embodiment;

FIG. 3 shows a computer system that is programmed or otherwise configured to implement methods provided herein, in accordance with an embodiment;

FIG. 4 schematically illustrates a system for predicting whether a subject has a behavioral or mental health condition using a natural language processing (“NLP”) model, in accordance with an embodiment;

FIG. 5 illustrates a process flow diagram for multilingual mental health screening, in accordance with an embodiment;

FIG. 6 illustrates a flow diagram of the pre-model strategy selector, in accordance with an embodiment;

FIG. 7 illustrates an example block diagram of the NLP models, in accordance with an embodiment;

FIG. 8 illustrates an example block diagram of the acoustic models, in accordance with an embodiment;

FIG. 9 illustrates a block diagram of the post-model strategy selector, in accordance with an embodiment;

FIG. 10 illustrates a block diagram of the feedback mechanism, in accordance with an embodiment; and

FIG. 11 depicts labels distribution for both English and Spanish test sets from a multilingual mental health screening experiment, in accordance with an embodiment.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### Overview

Disclosed is an end-to-end solution for predicting, detecting, or monitoring a behavioral or mental health condition. In particular, the system is configured to predict the behavioral or mental health condition of a speaker, regardless of the language or languages present in the speech. The received speech data from the speaker may include audio data or text data.

The system may make a language-independent prediction by leveraging a plurality of acoustic and natural language processing (NLP) models to process the speech. The models may be generic language models, multilingual models, or language-independent models.

The system may adapt to the input data and to new information produced during the machine learning process. For example, the system may use one or more machine learning models to first detect languages present in the speech. Then, a pre-model strategy selector may, based on at least the input data and possibly the detected languages, decide which machine learning algorithms to select to use to process the data. The pre-model strategy selector may choose to apply a joint or fused model derived from one or more acoustic or NLP models to process the input speech. Following analysis by the machine learning models, a post-model strategy selector may determine, based on a confidence score of the one or more models used and/or outputs of any of the models that may be used, to improve the prediction by taking remedial actions or using alternate models. Finally, the system may provide feedback to the subject or testing agent to improve the prediction performed, for example, through changing the interactions with the subject in the screening process or by using obtained knowledge for training purposes (e.g., training agents).

The system is designed to make predictions based on lexical and non-lexical or acoustic features in the input speech (which may be audio and/or text data), being able to operate effectively with speech from known or unknown languages, speech containing one or more languages (e.g., speech with code switching), or speech containing specific dialects, pidgins, or creoles.

Input speech may include audio or text data uploaded to a user device (e.g., of the subject, as a voice-based or text-based journal entry. The entry may or may not be multilingual in content. The disclosed system may perform analysis of the journal entry and determine a risk severity. Based on this risk severity, an application on the user device may suggest one or more recommended actions for the user. A recommended action may be a wellbeing or wellness exercise. The exercise may be related to cognitive behavioral therapy (CBT) or dialectical behavioral therapy (DBT). The application on the user device may provide therapy (e.g., talk or text-based therapy through the application) or may enable the user to contact a telehealth service.

One or more elements of the disclosed system may be integrated into or accessible through another system. For example, the analysis performed by the system may be accessible via an application programming interface (API) so that third-party developers may integrate the risk severity analysis into their own applications. These applications may include, for example, telehealth, text therapy, mental or behavioral health monitoring (e.g., remote monitoring), or other types of applications. Subjects may be analyzed periodically and their risk severity scores may be tracked, to chart a patient's progress over time.

### Description of Embodiments

Disclosed is a system that provides behavioral health prediction that may analyze a speech input that may be provided as audio or text data. The audio may be recorded from a human subject in real-time. The audio may be provided via recording. The text data may be input from a human subject in real time. The text data may be provided as a transcript. The audio data or text data may comprise speech from one or more of many languages. The behavior health prediction or mental health prediction may predict a health condition such as depression, anxiety, stress, schizophrenia, bipolar disorder, post-traumatic stress disorder (PTSD), or other disorders.

The one or more languages present in the speech data may include one or more of English, Spanish, Chinese (e.g., Mandarin, Wu, Yue, Jin, Xiang, Gan, or Cantonese), Hindi, Bengali, Portuguese, Russian, Japanese, Punjabi, Marathi, Telegu, Turkish, Korean, French, German, Vietnamese, Tamil, Urdu, Javanese, Italian, Arabic, Gujarati, Persian, Bhojpuri, Min, Hausa, Kannada, Indonesian, Polish, Yoruba, Malayalam, Odia, Maithili, Burmese, Sunda, Ukrainian, Igbo, Uzbek, Sindhi, Romanian, Tagalog, Dutch, Amharic, Pashto, Magahi, Thai, Saraiki, Khmer, Chhattisgarhi, Somali, Malaysian, Cebuano, Nepali, Assamese, Sinhalese, Kurdish, Fufulde, Bavarian, Azerbaijani, Greek, Chittagonian, Kazakh, Deccan, Hungarian, Albanian, Kinyarwanda, Danish, Finnish, Zulu, Rundi, Czech, Uyghur, Sylheti, Serbian, Croatian, Hungarian, Montenegrin, Haitian Creole, Swedish, Norwegian, Hmong, Xhosa, Belarusian, Konkani, Latin, Gaelic, Catalan, Occitan, Provencal, Maltese, Bosnian, Armenian, Slovenian, Hebrew, Yiddish, Aramaic, Ladino, Navajo, Native Hawaiian, Maori, Esperanto, Pig Latin, or another language or invented language. A language included in the speech data may be a creole (e.g., Haitian creole) or Pidgin language. A language included in the speech data may be regional variant or dialect (e.g., Louisiana French, Indian English).

The language may be a language that has been previously encountered by the system. Alternatively, the language may be a language that has not been previously encountered by the system.

The speech sample (audio or text) may be of any word length. For example, a text sample may be less than ten, less than 20, less than 30, less than 50, less than 100, less than 500, less than 1000, or less than 2000 words long. For example, a text sample may be greater than ten, greater than 20, greater than 30, greater than 50, greater than 100, greater than 500, greater than 1000, or greater than 2000 words long.

An audio sample of speech may be of any time length. For example, an audio sample may be less than ten, less than 20, less than 30, less than 50, less than 100, less than 500, less than 1000, or less than 2000 minutes long. For example, an audio sample may be greater than ten, greater than 20, greater than 30, greater than 50, greater than 100, greater than 500, greater than 1000, or greater than 2000 minutes long.

The input speech sample may be an active speech sample or a passive speech sample. An active speech sample may be a sample of speech taken from a subject in a clinical setting. For example, the speech may be recorded when the subject is being administered a survey by a human agent or an automated agent. In various embodiments, active speech samples are recorded when the subject knows that his or her speech elicitations are being recorded for the purpose of mental health screening. Passive speech may be conversational speech or observed speech when the subject is not being provided with a questionnaire or survey. For example, passive speech may be recorded from a conversation the subject is having with a health care provider or even a person not participating in the mental health screen.

Once the speech is collected, the system may identify one or more languages present in the speech sample. The system may include a software system or sub-system that may serve as a language detection unit (or “language detector”) to detect one or more languages present in the input speech sample. The machine learning algorithm may generate one or more probability scores indicating how likely the sample is to include multiple languages, each language corresponding to a probability score. For example, the machine learning algorithm may output a set of probabilities that show the sample has a 90% chance of including English, a 70% chance of including German, and a 3% chance of including Arabic. The language detection unit may apply labels to the input speech corresponding to the language or languages detected. The language detection unit may detect multiple languages present in the speech sample (i.e., code switching between languages). The language detection unit may detect particular dialects, pidgins, or creoles of one or more languages present.

The language detection unit may use metadata or demographic data to detect the languages present. For example, the language detection unit may be able to use as an input alongside with the speech audio or text data with information about the subject's age, gender, race, nationality or national origin, weight, medical history, address, occupation, marital status, socioeconomic status, education, number of children, other personal or personally identifiable information, or metadata which may include non-personally identifiable information. This information may include typing speed, ambient noise, testing context or environment, time delay in answering questions, vocal pitch, type of test administered, type of equipment used to obtain samples, duration of speech, audio quality, subject device, agent device, or other information. The metadata may include vital sign information, such as heart rate, blood glucose level, blood pressure, or other bodily measurements. The language detection unit may include an annotation unit to append labels to the speech samples.

The system may use a performance estimation unit to estimate, based at least in part on qualities of the input speech, how well one or more acoustic or NLP models may perform to predict a mental or behavioral health condition. For example, performance of the model may vary based on one or more languages present in the model, the quality of the text or acoustic signal (e.g., if the signal is incomplete or there is noise), the length of the signal, the type of the signal (acoustic, text, or both), acoustic features of the voice of the speaker. The performance estimation module may also consider the language identity distribution, the length of the audio session or signal, the topics in the audio session, the speech quality, the cadence or pitch of the subject speaking, what dialect of a particular language the speech belongs to, the speed of the speech, or the number of pauses of other vocal tics, when predicting performance of a particular model.

For example, a particular audio model may perform well on German speech but not English speech. A particular NLP model may perform well analyzing speech with a Cockney accent, but not well on speech with a Scottish accent. Another type of model may not perform well on a code-switched sample (e.g., an Indian speaker switching between Hindi and English).

The performance estimation unit (interchangeably used with “model performance estimator”) may use statistical modeling (e.g., Bayesian modeling) or machine learning to estimate the performance of one or more of the acoustic or NLP models.

The pre-model strategy selector determines how different combinations of acoustic and/or NLP models (e.g., using a weighted combination, an unweighted combination, a joint model, or model fusion) to use to predict, monitor, or detect the behavioral or mental health condition from the input speech (interchangeably referred to as “determining or selecting a strategy”). The pre-model strategy selector may determine the weighted combination using the input speech sample. In some embodiments, the pre-model strategy selector may use additional data. For example, the pre-model strategy selector may use language detection data or model performance data to determine whether to use a weighted combination of models, a joint model, or another configuration of models. In some embodiments, the pre-model strategy selector may use other data, such as demographic data or metadata. In some embodiments, along with acoustic and NLP models, the weighted combination of models may include demographic and/or metadata models. In some embodiments, the strategy selector may implement fusion models, or models that combine representations (e.g., latent representations) of outputs from one or more acoustic, NLP, or other models. The pre-model strategy selector may determine an optimal combination of models to best perform the prediction, monitoring, or detection task. An optimal combination may comprise maximizing precision, recall, a receiver operating characteristic (ROC) area under the curve (AUC), true positive rate, false positive rate, or other statistic associated with quantifying a performance of a machine learning algorithm or combination of machine learning algorithms. The pre-model strategy selector may determine whether feedback is to be incorporated into the mental or behavioral health screening process.

The pre-model strategy selector may devise the configuration of acoustic, NLP, and/or other models in one or more ways. The strategy selector may determine an order in which to implement the one or more acoustic, NLP, or other models. The strategy selector may determine magnitudes of weights to apply to each of the models selected. The strategy selector may determine how representations of data are to be fused. For example, representations may be fused using concatenation or max pooling. In addition, multimodal algorithms can be used as well.

The machine learning models may process the input data in accordance with the strategy selected by the pre-model strategy selector. These models may be supervised or unsupervised learning models. The models may be deep learning models. In some embodiments, the models include pre-processing algorithms to filter or package the input speech into data that is more suitable for machine learning analysis.

The models may be NLP models. The models may include generic language models. The models may include NLP translation models. The models may include multilingual NLP models. A model used by the system may have a similar architecture to another model used, or may have a different architecture from the other model. The models used may or may not be limited to the ones listed. The models may include transformers, such as multilingual machine translation (M2M100), multilingual bidirectional and auto-regressive transformer (mBART), multilingual text-to-text (mT5), cross-lingual robust bidirectional encoder representations from transformers (XLM-RoBERTa), Longformer, or a lite bidirectional encoder representations from transformers (ALBERT).

The models may be acoustic models. The models may include acoustic models trained on a single language. The acoustic models may be fine tuned on a single language. The models may include multilingual acoustic models (e.g. trained on acoustic data coming from different languages). The models may include language-independent acoustic models where features are selected or learned that does not depend on the language. For example, these features may include (but are not limited to include) pitch, formant frequency, speech rate, speaker-based embedding features such as i-vectors or x-vectors, and features learned from non-automatic speech recognition (ASR) tasks (e.g., sound classification) or learned from ASR tasks.

The models may be demographic or metadata models.

The models may be fusion models. The fusion models may combine output data (e.g., representations) from one or more of the acoustic, NLP, demographic, or metadata models. The output may be a score or a learned representation.

The system may process the input using a fused model derived from the plurality of models (or submodels) used (e.g., acoustic, NLP, fusion, or demographic). In such a scenario, each model selected may be trained independently. Then, the model outputs may be combined. The model outputs may be combined using concatenation, max pooling, or by another method, for example.

The system may process the input using a joint model derived from the plurality of models. In such a scenario, the models (or submodels), such as an acoustic model, an NLP model, or another model, are trained together or simultaneously to produce a joint model output.

The system may use a post-model strategy selector to compensate for low reliability from the predicted output from the combination of machine learning models. The reliability may be generated or calculated by one or more of the models themselves and provided to the post-model strategy selector. The post-model strategy selector may be used to update model parameters as predictions are being made, until a final prediction by the system is actionable. In cases of low reliability, the post-model strategy selector may perform one or more actions. The post-model strategy selector may prompt switching the language in which an agent administers the mental health screen. The post-model strategy selector may prompt human intervention (e.g., from a human agent such as a clinician or health care provider) in the screening process. The post-model strategy selector may use alternative models. The post-model strategy selector may change the prediction task (e.g., from depression to anxiety). The post-model strategy selector may, depending on model confidence and/or other factors, suggest one or more remedial actions to change the analysis or testing procedure.

The feedback mechanism may provide feedback to the agent (human or automated) providing the mental health screen to the subject, following completion of a mental health screen or selection of a pre-model strategy by the pre-model strategy selector. An automated agent may be an application (e.g., a smartphone application). The feedback mechanism may take as inputs results of the models or the post-model strategy selector and may output a recommended action. The feedback mechanism may be a rules-based mechanism or may use a statistical method or machine learning analysis. The feedback mechanism may, for example, prompt switching the language in which the test is conducted. The feedback mechanism may induce the patient to provide more speech input. The feedback mechanism may prompt the agent to ask follow-up questions. The feedback mechanism may take one or more actions to minimize a signal-to-noise ratio of the input sample. For example, the feedback mechanism may ask a patient to move to a quiet room or reconfigure the testing equipment. The feedback mechanism may determine that an incorrect question has been asked and prompt the agent to change the question.

### Acoustic Models

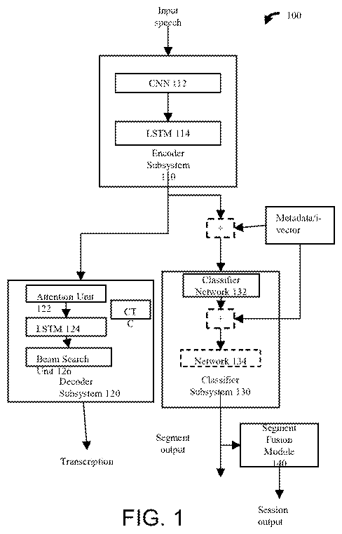

FIG. 1 schematically illustrates a system 100, in accordance with an embodiment. In other embodiments, acoustic models from differing architectures may be used. The system 100 can obtain the input speech through a microphone or array of microphones that are on an Internet-connected device or connected to an Internet-connected device (e.g., via a Bluetooth connection). The device may be a wearable device (e.g., a smart watch), a mobile phone, a tablet, a laptop computer, a desktop computer, smart speakers, a home assistance device (e.g., an Amazon Alexa® device or a Google Home® device), or the like. The device may have a mental health application. The mental health application may visually or audibly prompt the subject to answer questions about the subject's work and home life, sleep, mood, medical history, and the like. The subject's answers to the prompts may be used as the input speech. The system 100 may be implemented on the mobile application, and it may process the input speech locally on the subject's mobile device. Alternatively or additionally, the mobile device can transmit the speech to a remote location for processing. In some cases, the processing may be performed partially on the local device and partially on a remote server.

Alternatively or additionally, the input speech may be obtained via a clinical encounter with a health care professional. For example, an audio recording device may capture speech from the subject during a doctor's appointment. The doctor's appointment may be an in-person appointment or a telehealth appointment that is conducted remotely. Other contexts may include telephone, online, in-office or in-home appointments with providers, care teams, or case managers.

The subsystems of FIG. 1 may be implemented on one or more computing devices. The computing devices may be servers, desktop or laptop computers, electronic tablets, mobile devices, smart speakers, smart watches, wearable devices, or the like. The computing devices may be located in one or more locations. The computing devices may have general-purpose processors, graphics processing units (GPU), application-specific integrated circuits (ASIC), field-programmable gate-arrays (FPGA), machine learning accelerators, or the like. The computing devices may additionally have memory, e.g., dynamic or static random-access memory, read-only memory, flash memory, hard drives, or the like. The memory may be configured to store instructions that, upon execution, cause the computing devices to train the system 100 or predict whether a subject has a behavioral or mental health state of interest. The computing devices may additionally have network communication devices. The network communication devices can enable the computing devices to communicate with each other and with any number of user devices, over a network. For example, the network communication devices may allow the computing devices that implement the system 100 to communicate with health care professionals' mobile devices regarding the predicted behavioral or mental health states of subjects. The network may be a wired or wireless network. For example, the network may be a fiber optic network, Ethernet® network, a satellite network, a cellular network, a Wi-Fi® network, a Bluetooth® network, or the like. In other implementations, the computing devices may be several distributed computing devices that are accessible through the Internet. Such computing devices may be considered cloud computing devices.

### NLP Model Parameters

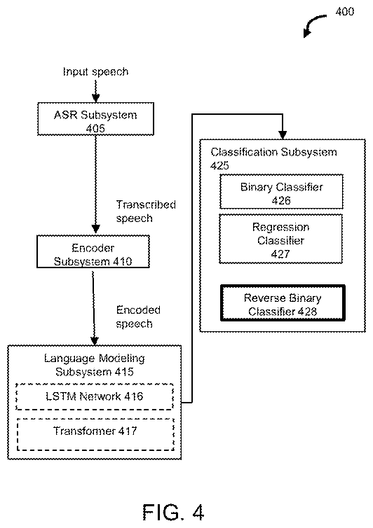

FIG. 4 schematically illustrates a system 400 for predicting whether a subject has a behavioral or mental health condition using an NLP model. The system 400 may include an automatic speech recognition (“ASR”) subsystem 405, an encoder subsystem 410, a language model subsystem 415, and a classification subsystem 425. The ASR subsystem 405 can generate a transcription of input speech from the subject. In some cases, the ASR subsystem 405 may include a third-party ASR model, e.g., Google ASR. The third-party ASR may be a 1-best hypothesis ASR, or it may take into account word uncertainty or may include word confusion information. In other cases, the ASR subsystem 405 may include a custom ASR model.

The subsystems of FIG. 4 and their components may be implemented on one or more computing devices. The computing devices may be servers, desktop or laptop computers, electronic tablets, mobile devices, or the like. The computing devices may be located in one or more locations. The computing devices may have general-purpose processors, graphics processing units (GPU), application-specific integrated circuits (ASIC), field-programmable gate-arrays (FPGA), or the like. The computing devices may additionally have memory, e.g., dynamic or static random-access memory, read-only memory, flash memory, hard drives, or the like. The memory may be configured to store instructions that, upon execution, cause the computing devices to implement the functionality of the subsystems. The computing devices may additionally have network communication devices. The network communication devices may enable the computing devices to communicate with each other and with any number of user devices, over a network. The network may be a wired or wireless network. For example, the network may be a fiber optic network, Ethernet® network, a satellite network, a cellular network, a Wi-Fi® network, a Bluetooth® network, or the like. In other implementations, the computing devices may be several distributed computing devices that are accessible through the Internet. Such computing devices may be considered cloud computing devices.

### System Outputs

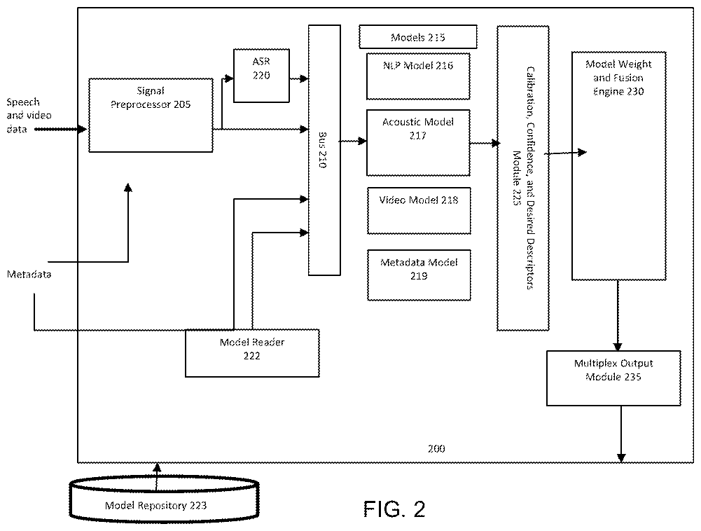

The system 200 of FIG. 2 can output an electronic report identifying whether a patient is at risk of a mental or behavioral condition. The electronic report can be configured to be displayed on a graphical user interface of an electronic device. The electronic device may belong to a patient, subject, health care provider, payor, care team, a family member of the subject or patient, or another stakeholder. The electronic report can include a quantification of the risk of the mental or physiological condition, e.g., a normalized score. The score can be normalized with respect to the entire population or with respect to a sub-population of interest. The electronic report can also include a confidence level of the normalized score. The confidence level can indicate the reliability of the normalized score (i.e., the degree to which the normalized score can be trusted).

The electronic report can include visual graphical elements. For example, if the patient has multiple scores from multiple screening or monitoring sessions that occurred at several different times, the visual graphical element may be a graph that shows the progression of the patient's scores over time.

The system 200 can output the electronic report to the patient or a contact person associated with the patient, a healthcare provider, a healthcare payer, or another third-party. The system 200 can output the electronic report substantially in real-time, even while the screening, monitoring, or diagnosis is ongoing. In response to a change in the normalized score or confidence during the course of the screening, monitoring, or diagnosis, the electronic report can be updated substantially in real-time and be re-transmitted to the user.

In some cases, the electronic report may include one or more descriptors about the patient's mental state. The descriptors can be a qualitative measure of the patient's mental state (e.g., “mild depression”). Alternatively or additionally, the descriptors can be topics that the patient mentioned during the screening. The descriptors can be displayed in a graphic, e.g., a word cloud.

The models described herein may be optimized for a particular purpose or based on the entity that may receive the output of the system. For example, the models may be optimized for sensitivity in estimating whether a patient has a mental condition. Healthcare payers such as insurance companies may prefer such models so that they can minimize the number of insurance payments made to patients with false positive diagnoses. In other cases, the models may be optimized for specificity in estimating whether a patient has a mental condition. Healthcare providers may prefer such models. The system may select the appropriate model based on the stakeholder to which the output will be transmitted. After processing, the system can transmit the output to the stakeholder.

The models described herein can alternatively be tuned or configured to process speech and other data according to a desired level of sensitivity or a desired level of specificity determined by a clinician, healthcare provider, insurance company, or government regulated body. Alternatively or additionally, the models can be tuned, configured, or trained to optimize precision, recall, F1, equal error rate (“EER”), positive predictive value (“PPV”), negative predictive value (“NPV”), likelihood ratio positive (“LR+”), likelihood ratio negative (“LR-”), concordance correlation coefficient (“CCC”), Pearson correlation coefficient (“PCC”), root mean squared error (“RMSE”), mean absolute error (“MAE”), or any other relevant performance metric.

The electronic report may include a “word cloud” or “topic cloud” extracted from a text transcript of the patient's speech. A word cloud may be a visual representation of individual words or phrases, with words and phrases used most frequently designated using larger font sizes, different colors, different fonts, different typefaces, or any combination thereof. Depicting word or phrase frequency in such a way may be helpful as depressed patients commonly say particular words or phrases with larger frequencies than non-depressed patients. For example, depressed patients may use words or phrases that indicate dark, black, or morbid humor. They may talk about feeling worthless or feeling like failures, or use absolutist language, such as “always”, “never”, or “completely.” Depressed patients may also use a higher frequency of first-person singular pronouns (e.g., “I”, “me”) and a lower frequency of second- or third-person pronouns when compared to the general population. The system may be able to train a machine learning algorithm to perform semantic analysis of word clouds of depressed and non-depressed people, in order to be able to classify people as depressed or not depressed based on their word clouds. Word cloud analysis may also be performed using unsupervised learning. For example, the system may analyze unlabeled word clouds and search for patterns, in order to separate people into groups based on their mental states. The generated words may indicate a decreased or increased risk of depression (i.e., that are associated with an increased or decreased risk of depression).

Similarly, the electronic report may include predicted personality traits of the patient. Personality traits (e.g., introversion or extroversion) can be inferred from speech length.

The electronic report may additionally include evidence-based psychoeducation materials and support strategies. The materials and support strategies may be tailored to the patient's score. The materials and support strategies may be provided directly to the patient in the form of videos, text, and assignments, or the materials and support strategies may be provided to the patient's health care provider, who can lead the psychoeducation process.

### Use Cases

The disclosed health screening system described herein may be used to assist with treatment of patients in regions of the world where mental health infrastructure is limited. The system may provide a central infrastructure to manage health screening of patients or subjects from many countries. Additionally, migration due to natural disasters, climate change, terrorism and other factors may cause mass immigration or movement of people into areas in which native people speak different languages. Because the models used by the system may be variously language-agnostic, language-independent, or multilingual, the system disclosed may be capable of assisting patients in a wide variety of regions, communities, and contexts, whom had been previously underserved.

Additionally, the system may be used to test employees who have emotionally taxing or high-stress jobs for mental fitness. Clinicians or algorithms may be used to develop screening or monitoring methods for these conditions. For example, the system may base an assessment off of queries similar to those tested in the Minnesota Multiphasic Personality Inventory (MMPI) and MMPI-2.

The system may also be used to screen military personnel for mental fitness. For example, the system may implement an assessment that uses queries with similar subject matter to those asked on the Primary Care Post-Traumatic Stress Disorder for Diagnostic and Statistical Manual of Mental Disorders (DSM)-5 (PC-PTSD-5) to test for PTSD. In addition to PTSD, the system may screen military personnel for depression, panic disorder, phobic disorder, anxiety, and hostility. The system may employ different surveys to screen military personnel pre- and post-deployment. The system may segment military personnel by segmenting for occupation, and segment military personnel by branch, officer or enlisted, gender, age, ethnicity, number of tours/deployments, marital status, medical history, and other factors.

The system may be used to evaluate prospective gun buyers, e.g., by implementing background checks. Assessments may be designed, by clinicians or algorithmically, to evaluate prospective buyers for mental fitness for owning a firearm. The survey may have a requirement to determine, using questions and follow-up questions, if a prospective gun buyer would be able to be certified as a danger to him or herself or others, by a court or other authority.

### Scoring

The models described herein may produce scores at various stages of a mental or behavioral health assessment. The scores produced may be scaled scores or binary scores. Scaled scores may range over a large number of values, while binary scores may be one of two discrete values. The models may interchange binary and scaled scores at various stages of the assessment, to monitor different mental states, or update particular binary scores and particular scaled scores for particular mental states over the course of an assessment.

The scores produced by the system, either binary or scaled, may be produced after each response to each query in the assessment, or may be formulated in part based on previous queries. In the latter case, each marginal score acts to fine-tune a prediction of depression, or of another mental state, as well as to make the prediction more robust. Marginal predictions may increase confidence measures for predictions of mental states in this way, after a particular number of queries and responses (correlated with a particular intermediate mental state)

For scaled scores, the refinement of the score may allow clinicians to determine, with greater precision, severities of one or more mental states the patient is experiencing. For example, the refinement of the scaled score, when observing multiple intermediate depression states, may allow a clinician to determine whether the patient has mild, moderate, or severe depression. Performing multiple scoring iterations may also assist clinicians and administrators in removing false negatives, by adding redundancy and adding robustness. For example, initial mental state predictions may be noisier, because relatively fewer speech segments are available to analyze, and NLP algorithms may not have enough information to determine semantic context for the patient's recorded speech. Even though a single marginal prediction may itself be a noisy estimate, refining the prediction by adding more measurements may reduce the overall variance in the system, yielding a more precise prediction. The predictions described herein may be more actionable than those which may be obtained by simply administering a survey, as people may have incentive to lie about their conditions. Administering a survey may yield high numbers of false positive and false negative results, enabling patients who need treatment to slip through the cracks. In addition, although trained clinicians may notice voice and face-based biomarkers, they may not be able to analyze the large amount of data the models disclosed herein are able to analyze.

The scaled score may be used to describe a severity of a mental state. The scaled score may be, for example, a number between 1 and 5, or between 0 and 100, with larger numbers indicating a more severe or acute form of the patient's experienced mental state. The scaled score may include integers, percentages, or decimals. Conditions for which the scaled score may express severity may include, but are not limited to depression, anxiety, stress, PTSD, phobic disorder, schizophrenia, and panic disorder. In one example, a score of 0 on a depression-related aspect of an assessment may indicate no depression, a score of 50 may indicate moderate depression, and a score of 100 may indicate severe depression. The scaled score may be a composition of multiple scores. A mental state may be expressed as a composition of mental sub-states, and a patient's composite mental state may be a weighted average of individual scores from the mental sub-states. For example, a composition score of depression may be a weighted average of individual scores for anger, sadness, self-image, self-worth, stress, loneliness, isolation, and anxiety.

A scaled score may be produced using a model that uses a multilabel classifier. This classifier may be, for example, a decision tree classifier, a k-nearest neighbors classifier, or a neural network-based classifier. The classifier may produce multiple labels for a particular patient at an intermediate or final stage of assessment, with the labels indicating severities or extents of a particular mental state. For example, a multilabel classifier may output multiple numbers, which may be normalized into probabilities using a softmax layer. The label with the largest probability may indicate the severity of the mental state experienced by the patient.

The scaled score may also be determined using a regression model. The regression model may determine a fit from training examples that are expressed as sums of weighted variables. The fit may be used to extrapolate a score from a patient with known weights. The weights may be based in part on features, which may be in part derived from the audiovisual signal (e.g., voice-based biomarkers) and in part derived from patient information, such as patient demographics. Weights used to predict a final score or an intermediate score may be taken from previous intermediate scores.

The scaled score may be scaled based on a confidence measure. The confidence measure may be determined based on recording quality, type of model used to analyze the patient's speech from a recording (e.g., audio, visual, semantic), temporal analysis related to which model was used most heavily during a particular period of time, and the point in time of a specific voice-based biomarker within an audiovisual sample. Multiple confidence measures may be taken to determine intermediate scores. Confidence measures during an assessment may be averaged in order to determine a weighting for a particular scaled score.

The binary score may reflect a binary outcome from the system. For example, the system may classify a user as being either depressed or not depressed. The system may use a classification algorithm to do this, such as a neural network or an ensemble method. The binary classifier may output a number between 0 and 1. If a patient's score is above a threshold (e.g., 0.5), the patient may be classified as “depressed.” If the patient's score is below the threshold, the patient may be classified as “not depressed.” The system may produce multiple binary scores for multiple intermediate states of the assessment. The system may weight and sum the binary scores from intermediate sates of the assessment in order to produce an overall binary score for the assessment.

The outputs of the models described herein can be converted to a calibrated score, e.g., a score with a unit range. The outputs of the models described herein can additionally or alternatively be converted to a score with a clinical value. A score with a clinical value can be a qualitative diagnosis (e.g., high risk of severe of depression). A score with a clinical value can alternatively be a normalized, qualitative score that is normalized with respect to the general population or a specific sub-population of patients. The normalized, qualitative score may indicate a risk percentage relative to the general population or to the sub-population.

The systems described herein may be able to identify a mental state of a subject (e.g., a mental disorder or a behavioral disorder) with less error (e.g., 10% less) or a higher accuracy (e.g., 10% more) than a standardized mental health questionnaire or testing tool. The error rate or accuracy may be established relative to a benchmark standard usable by an entity for identifying or assessing one or more medical conditions comprising the mental state. The entity may be a clinician, a healthcare provider, an insurance company, or a government-regulated body. The benchmark standard may be a clinical diagnosis that has been independently verified.

### Model-Specific Confidence Measures

The models described herein may use confidence measures. A confidence measure may be a measure of how effective the score produced by the machine learning algorithm may be in order of accurately predicting a mental state, such as depression. A confidence measure may depend on conditions under which the score was taken. A confidence measure may be expressed as a whole number, a decimal, or a percentage. Conditions may include a type of recording device, an ambient space in which signals were taken, background noise, patient speech idiosyncrasies, language fluency of a speaker, the length of responses of the patient, an evaluated truthfulness of the responses of the patient, and frequency of unintelligible words and phrases. Under conditions where the quality of the signal or speech makes it more difficult for the speech to be analyzed, the confidence measure may have a smaller value. In some embodiments, the confidence measure may be added to the score calculation, by weighting a calculated binary or scaled score with the confidence measure. In other embodiments, the confidence measure may be provided separately. For example, the system may tell a clinician that the patient has a 0.93 depression score with 75% confidence.

The confidence level may also be based on the quality of the labels of the training data used to train the models that analyze the patient's speech. For example, if the labels are based on surveys or questionnaires completed by patients rather than official clinical diagnoses, the quality of the labels may be determined to be lower, and the confidence level of the score may thus be lower. In some cases, it may be determined that the surveys or questionnaires have a certain level of untruthfulness. In such cases, the quality of the labels may be determined to be lower, and the confidence level of the score may thus be lower.

Various measures may be taken by the system in order to improve a confidence measure, especially where the confidence measure is affected by the environment in which the assessment takes place. For example, the system may employ one or more signal processing algorithms to filter out background noise or use impulse response measurements to determine how to remove effects of reverberations caused by objects and features of the environment in which the speech sample was recorded. The system may also use semantic analysis to find context clues to determine the identities of missing or unintelligible words.

In addition, the system may use user profiles to group people based on demeanor, ethnic background, gender, age, or other categories. Because people from similar groups may have similar voice-based biomarkers, the system may be able to predict depression with higher confidence, as people who exhibit similar voice-based biomarkers may indicate depression in similar manners.

For example, depressed people from different backgrounds may be variously categorized by slower speech, monotone pitch or low pitch variability, excessive pausing, vocal timbre (gravelly or hoarse voices), incoherent speech, rambling or loss of focus, terse responses, and stream-of-consciousness narratives. These voice-based biomarkers may belong to one or more segments of patients analyzed.

### Multilingual Mental Health Screening

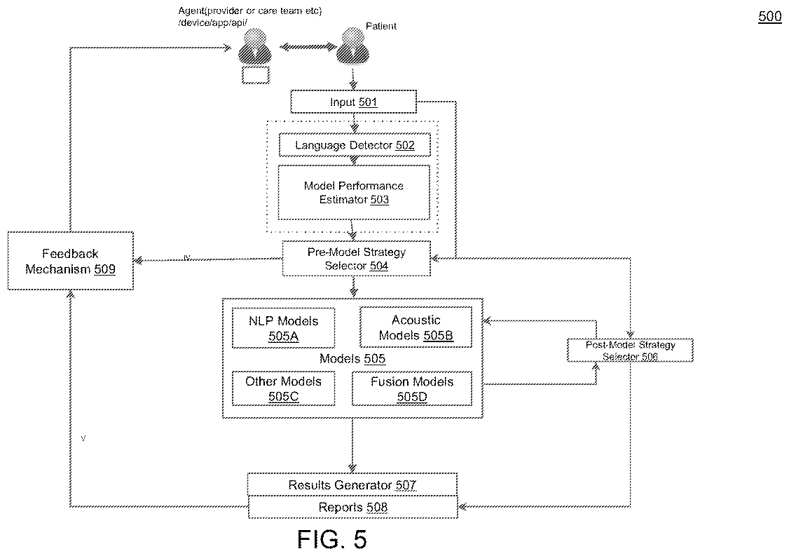

FIG. 5 illustrates a process flow diagram 500 for multilingual mental health screening, in accordance with an embodiment. A patient is administered a test by an agent or test provider, which may be a human test provider (e.g., a physician or clinician) or an automated testing interface. The automated testing interface may administer a survey with questions designed to elicit speech in audio or textual form as input 501. The multilingual health screening process performs a plurality of operations on the input speech 501 in order to monitor, detect, or predict mental or behavioral health conditions. The process is adaptive and may take measurements of the predictive process at multiple stages in order to fine-tune the prediction.

The input 501 may be an audio or text input. An audio input may include one or more speech or conversation snippets or elicitations. The elicitations may each be a few seconds or a few minutes long. The input may be text elicited from the subject and recorded. The input may also be a text transcript of audio produced by the subject. Text inputs may be of any length (e.g., from a few characters to many pages in length). Audio inputs may be of any duration (e.g., from a few seconds to many hours).

The language detector 502 may determine one or more languages present in the input speech 501. The language detector 502 may produce a probability distribution indicating which languages are likely to be present in the sample. This distribution may indicate, for monolingual speech, candidate languages the speech may belong to. For a multilingual sample (e.g., a sample containing code switching), the language detector 502 may indicate which languages are present. If a language is unknown to the system, the language selection analysis may select a most similar known language from the languages it has been trained to recognize.

The model performance estimator 503 may determine performance estimates for different models 505 for a given piece of speech in a given language. This estimates may then be used by pre-model strategy selector 504 to select a subset of models to compute a score associated with a mental health risk. In some embodiments, pre-model strategy selector 504 may not process the speech if it determines that no model can effectively process the provided speech input. In such cases, the system would implement feedback mechanism 509 upon making such a determination. For multilingual speech, the model performance estimator 503 may produce multiple confidence values for the multiple languages present.

In some embodiments, the language detector 502 and model performance estimator 503 are not present. In these embodiments, the pre-model strategy selector 504 may rely more heavily on the input 502 and feedback from feedback mechanism 509 before devising a strategy.

The pre-model strategy selector 504 may select a subset of models 505 (or model configuration) to use to make the mental health prediction (e.g., using joint modeling or model fusion). The pre-model strategy selector may use the input data 501 along with the feedback from feedback mechanism 509 and language information from the language detector 502 and model performance estimator 503 to determine the subset of machine learning models 505 which will generate the mental health prediction.

The pre-model strategy selector 504 may choose models 505 to use based on the availability of resources. For example, the pre-model strategy selector 504 may decide to use a translation model if automated speech recognition (ASR) is available to during the mental health screening.

The pre-model strategy selector 504 may train new models or may adapt by collecting labels or population statistics. The pre-model strategy selector 504 can train the models with the population statistics or direct labels.

The pre-model strategy selector 504 may drive the model selection strategy by using one or more of the following: a latency of the prediction (e.g., single model vs. multiple, automated speech recognition, language detection, or depression detection), the context in which the input 501 was collected (human-device, human-machine, etc.), business flow requirements, a monetary cost for using a particular model, or demographic data that may determine a data flow path. The pre-model strategy selector may determine whether the configuration of acoustic or NLP models is unreliable, and if so, may use the feedback mechanism 509 to provide one or more remedial measures to improve the mental health screen.

The models 505 may be NLP models 505A, acoustic models 505B, other models (e.g., demographic, metadata) 505C, or fusion models 505D. The models may use machine learning algorithms to generate intermediate outputs. These intermediate outputs may be weighted and combined according to the instructions from the pre-model strategy selector 504.

The NLP models 505A may be translation-based models. These models may be trained on pairs of sentences (e.g., English-Spanish, Spanish-English, English-Portuguese. These models may require less data for training than the generic models. These models may include many-to-many architectures which translate among 100 languages (M2M100) architectures.

The NLP models 505A may be multilingual models. These models may be trained on large volumes of data on many languages at a time. Multilingual models may be based on multilingual text-to-text transfer transformer (mT5) model architectures.

Any of the NLP models 505A or acoustic models 505B may be fine-tuned to particular tasks, e.g., to detect particular health conditions. Fine tuning may involve training generic models using data from a specific domain, and then performing classification or regression supervised training.

The post-model strategy selector 506 may determine whether the models 505 produce an unreliable result. The post-model strategy selector 506, based on information from the input 501, pre-model strategy selector 504, or models 505, may select one or more remedial actions to improve the prediction. These actions may be performed iteratively until the prediction result produced by the models 505 is sufficiently reliable or actionable. Remedial actions performed by the post-model strategy selector 506 may include switching the language, asking for human intervention, using a prior (e.g., instead of using the output from the models 505, using a distribution of PHQ scores across particular demographics to provide screening results), using one or more alternative models 505, or predicting different metrics. The post-model strategy selector 506 may iteratively induce remedial actions until a reliability threshold is met or exceeded.

Results generator 507 may generate one or more reports 508 from the outputs of models 505. The results 508 may be electronic reports that include one or more charts or graphs describing the outputs of models 505. The results may be viewed by the agent or by the subject. The results 508 of the models 505 may be used to calculate additional scores or metrics to be used by clinicians. The results provided by results generator 507 may be related to remedial actions provided by the post-model strategy selector 506.

The feedback mechanism 509 may use information from the results 508 and/or the pre-model strategy selector 504 to recommend changes to the mental health screening procedure.

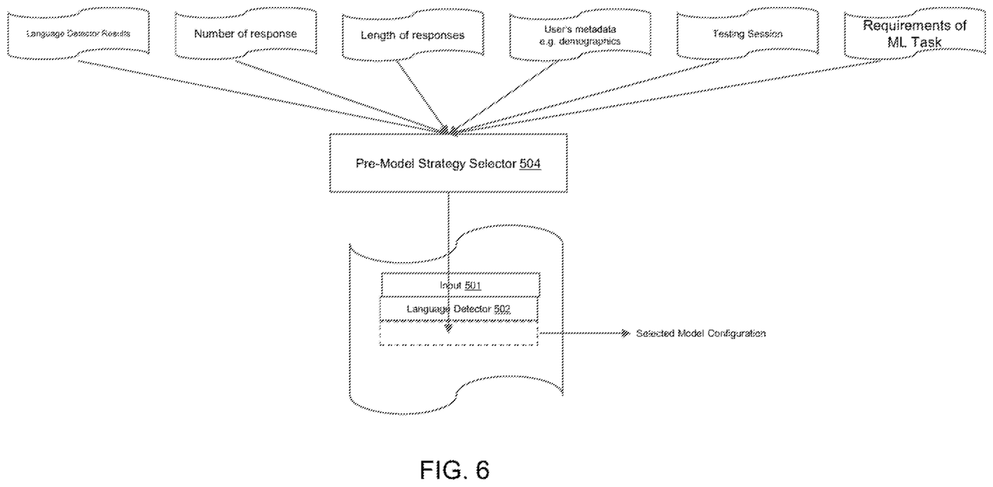

FIG. 6 illustrates a flow diagram of the pre-model strategy selector 504. The pre-model strategy selector 504 may take as an input the language detection results, which may include whether the input is a mother tongue. The inputs to the pre-model strategy selector 504 may include the number of responses, the length of responses, the user's metadata (e.g., demographics), and information about the closed loop session between the patient and the provider or the automated testing module.

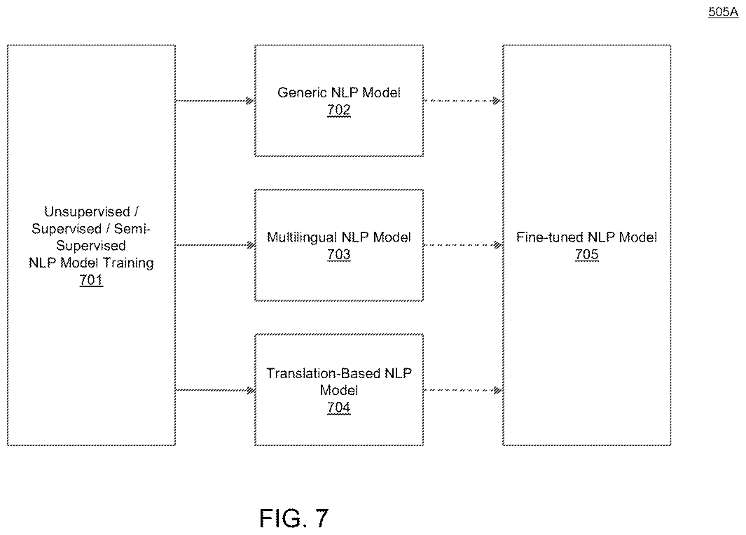

FIG. 7 illustrates an example block diagram of the NLP models 505A, in accordance with an embodiment. The models 505A may be trained 701. Then, generic NLP models 702, multilingual NLP models 703, and translation-based NLP models 704 may operate on input 501 to produce intermediate outputs, which may be fine-tuned by fine-tuned NLP model 705.

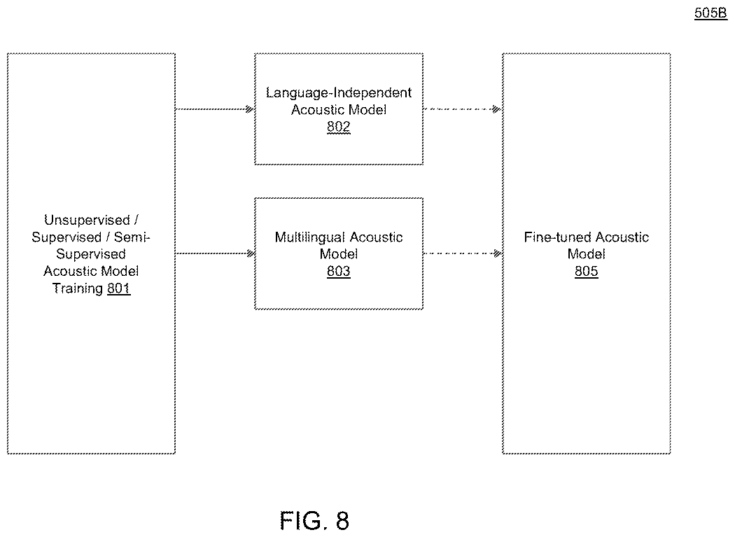

FIG. 8 illustrates an example block diagram of the acoustic models 505B, in accordance with an embodiment. The models 505A may be trained 801. Then, language-independent acoustic models 802 and multilingual acoustic models 803 may operate on input 501 to produce intermediate outputs, which may be fine-tuned by fine-tuned acoustic models 805.

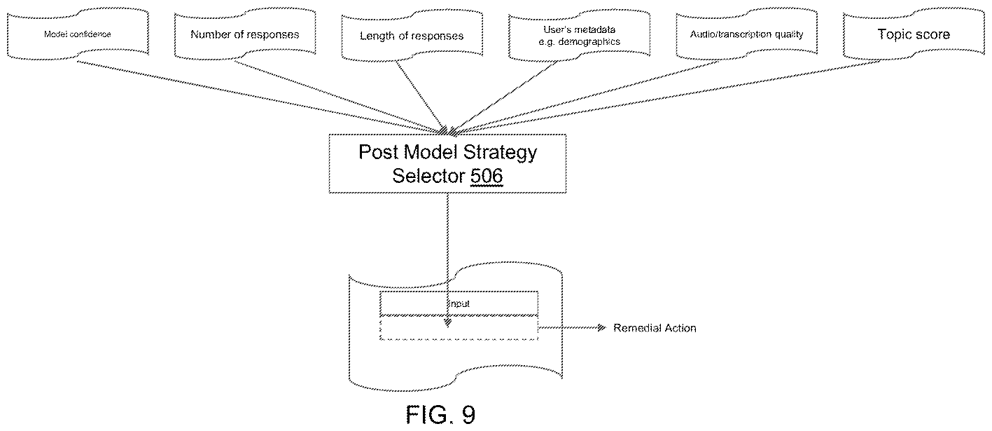

FIG. 9 illustrates a block diagram of the post-model strategy selector 506, in accordance with an embodiment. The post-model strategy selector 506 may take as inputs model confidence, a number of responses, a length of responses, a user's metadata (e.g., demographics, personal or non-personal information), an audio/transcription quality, topic score. The post-model strategy selector 506 may provide its results as input for future testing actions.

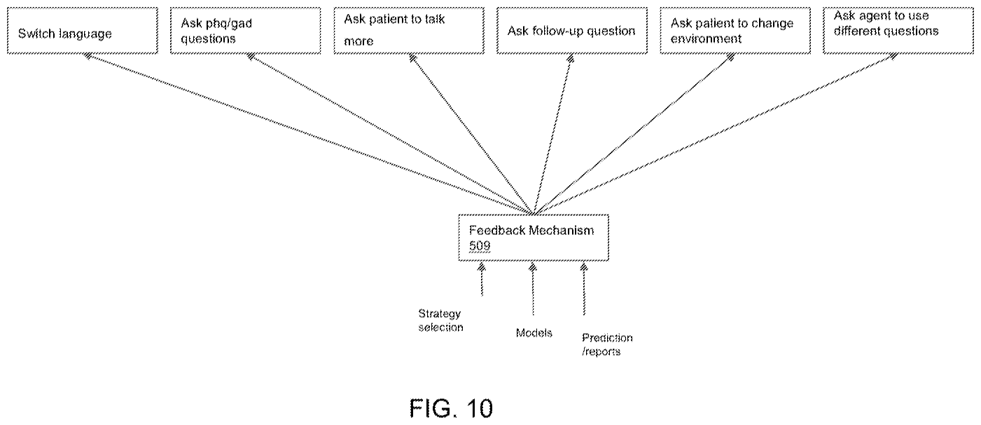

FIG. 10 illustrates a block diagram of the feedback mechanism 509, in accordance with an embodiment. The feedback may be based on the strategy selection 504, the models 505, or other final reports, or a combination of the preceding elements. The feedback mechanism 509 may prompt the testing model to switch the language the screen is administered in or ask one or more questions from the PHQ/GAD questionnaire. The feedback mechanism 509 may prompt the patient to provide more speech elicitations. The feedback mechanism 509 may prompt the system to ask a follow up question. The feedback mechanism 509 may prompt the patient to perform one or more actions to increase the SNR of the speech signal, including moving to a quieter room to perform the screening. The feedback mechanism 509 may prompt the testing agent to use a different set of questions.

### Quality Control

Situations may arise in which input speech provided by a patient is not acceptable. In such cases, the systems described herein can flag the input speech in real time. In one example, a compliant user may be unable to produce speech or may produce it at suboptimal quality or quantity. An acoustic quality detector can analyze speech as it is collected and generate a warning in real-time if the quality of the speech (e.g., its volume) is too low. The system can also determine word counts in real time and can supply a new set of prompts if the word counts are not high enough. The new prompts may be designed to elicit longer or more responses. In another example, a user may try to game the system (e.g., to obtain an incentive or avoid a diagnosis). For such users, an ASR model can process the speech to determine if it is significantly different from speech from “good” users. Input from test users is then compared to this model in real time to see if the word patterns are too far from what is expected from good users. This approach can catch users who play audio from another source instead of speaking live to the system, or who talk but do not attempt to talk about the questions asked. The system can then present an alert to the user or tag the audio files.

### Non-Speech Models

In some cases, the systems described herein may include non-speech models, including breathing models, laughter models, demographic models, metadata models, and pause models. Modeling breathing may be useful for predicting anxiety or mania. Modeling laughter (or an absence thereof) may be useful for predicting depression. Pauses may also be indicative of certain behavioral or mental health conditions. The outputs of non-speech models can be fused with the outputs of the acoustic model.

### Computer Systems

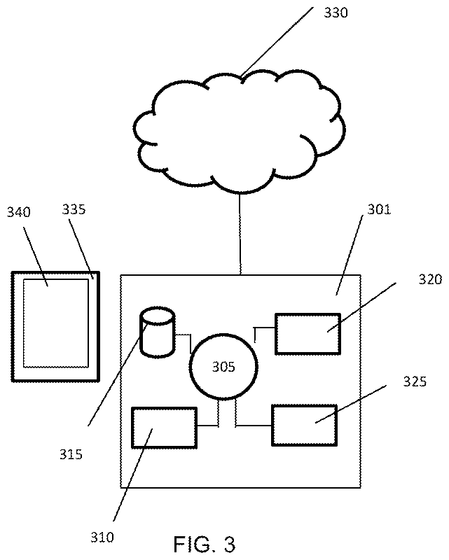

The present disclosure provides computer systems that are programmed to implement methods of the disclosure. FIG. 3 shows a computer system 301 that is programmed or otherwise configured to implement the system 500 of FIG. 5 or perform the training processes described herein.

The computer system 301 includes a central processing unit (CPU, also “processor” and “computer processor” herein) 305, which may be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 may be a data storage unit (or data repository) for storing data. The computer system 301 can be operatively coupled to a computer network (“network”) 330 with the aid of the communication interface 320. The network 330 may be the Internet, an internet and/or extranet, or an intranet and/or extranet that is in communication with the Internet. The network 330 in some cases is a telecommunication and/or data network. The network 330 may include one or more computer servers, which can enable distributed computing, such as cloud computing. The network 330, in some cases with the aid of the computer system 301, can implement a peer-to-peer network, which may enable devices coupled to the computer system 301 to behave as a client or a server.

The CPU 305 can execute a sequence of machine-readable instructions, which may be embodied in a program or software. The instructions may be stored in a memory location, such as the memory 310. The instructions can be directed to the CPU 305, which can subsequently program or otherwise configure the CPU 305 to implement methods of the present disclosure. Examples of operations performed by the CPU 305 may include fetch, decode, execute, and writeback.

The CPU 305 may be part of a circuit, such as an integrated circuit. One or more other components of the system 301 may be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases may include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 301 via the network 330.

Methods as described herein may be implemented by way of machine (e.g., computer processor) executable code stored on an electronic storage location of the computer system 301, such as, for example, on the memory 310 or electronic storage unit 315. The machine executable or machine-readable code may be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processer adapted to execute the code or can be compiled during runtime. The code can be supplied in a programming language that may be selected to enable the code to execute in a pre-compiled or as-compiled fashion.

Aspects of the systems and methods provided herein, such as the computer system 301, may be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, Blu-ray, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 may include or be in communication with an electronic display 335 that comprises a user interface (UI) 340 for providing, for example, one or more queries to a user that may elicit speech from the user. Examples of UI's include, without limitation, a graphical user interface (GUI) and web-based user interface.

Methods and systems of the present disclosure may be implemented by way of one or more algorithms. An algorithm may be implemented by way of software upon execution by the central processing unit 305. The algorithm may, for example, be any of the acoustic models, machine learning models, or training processes described herein.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Examples

### Speech-Based Depression Prediction Experiment

This section explains a particular experiment and should not be construed to limit other sections of this disclosure.

Speech-based depression prediction is important for future healthcare applications, but it may be difficult to port to new languages because of the need for large amounts of labeled training data. This experiment explores two novel alternative NLP approaches for depression prediction in a new language (Spanish), using labeled training data from only an existing language corpus (English). Both methods affect only the test data, not the training data. English data (65,000 responses, train and test) and Spanish data (1,600 responses, test only) come from automatically transcribed spoken responses in similar behavioral health screening applications. The Machine Translation approach performs Spanish-to-English translations of the Spanish test data using an English “A Lite Bidirectional Encoder Representations from Transformers” (ALBERT) model. The multilingual approach uses an multilingual text-to-text transfer transformer (mT5) model, in which a model is built from parallel text in over 100 languages without any direct explicit translation. Results show that both approaches achieve strong classification results compared to results on English train/test only, with slightly better results for the machine translation than the multilingual method.

### Introduction and Background

The incidence of depression has been widely spread across the world and has been constantly growing over the last decade. Because of under-diagnosed characteristics associated with mental health conditions, there is tremendous pressure to utilize technology in this space in particular for screening. The widely used Patient Health Questionnaire (PHQ-9) for depression screening lacks scalability and is difficult to use to assist patients when their cognitive capabilities are not sufficient to do it independently. Conversational analysis in the mental health domain is another example of measuring or screening depression condition. Various publications indicate that such an approach may be effective.

Digital technology may be used to create an alternative to the PHQ-9 by enabling analysis of a patient's speech (either recorded as audio or transcribed as text, or both).

This disclosure focuses on natural language analysis. From statistical word occurrences, through an n-grams approach, word embeddings, language models and transfer learning, the advancements in NLP have allowed to significantly outperform preceding state of the art NLP benchmarks.

A few trends are emerging in the NLP field. One of these is transfer learning, which enables achieving much better model performance results on various downstream NLP tasks by building generic deep learning networks that are taught in most of the cases, to predict some word occurrences in a given text passages (e.g. following word, word masking, or others). Another one is the progress in deep learning architectures in which transformer class language models have been successful recently. A third factor contributing to the latest breakthroughs in the NLP space is data availability. Large corpora may help to make models robust. Unfortunately, data for all languages is not widely available. English is the most dominant language in the machine learning space and generic, non-subject specific data is extensively available. But for some other languages and NLP downstream tasks, data acquisition still remains a challenge. In the past, there have been attempts to train a separate language model for each language. Latest advancements allow for a multilingual pretrained model that can be used for the downstream tasks that should compensate for the lack of downstream labeled data.

The mental health screening domain is not an exception to the problem of data not being widely available. It may be difficult to obtain a large language corpora, slowing down the process of technology adoption e.g. human to machine depression detection.

This disclosure concerns whether it is possible to train a classification language model on English labelled depression data and evaluate depression condition on Spanish data. This experiment analyzes the ability of the English corpora to be used as a training set for predicting depression conditions on a Spanish language test set. Thus, the following labelled data resources are going to be used—train and test set of English text as well as Spanish test set. In terms of methodology, the disclosure proposes a comparison of two most intuitive scenarios. The first scenario is to translate Spanish data (automatically) to English and evaluate on an English language model. The second, is to train the multilingual model, optimize it for English corpora and evaluate it on both English and Spanish evaluation sets.

### Method

### Data

The disclosure uses an American English corpora and a Spanish corpora as speech sets. Both speech sets were collected in the same manner. Otherwise, language different data would be confounded with speech elicitation methodology. In both of the corpora, subjects were incentivized financially. The users interacted with an app that posed questions on different topics such as “concerns” and “home life”. Users answered by speaking freely. Corpus statistics are given in Table 1. The large English Population (GP) corpus contains over 15,000 (65,000 responses) sessions of speech. Training and testing partitions may contain no overlapping speakers. Users range in age from 18-65, with a mean age of 30. Spoken responses average about 160 words. Users responded to 4-6 (mean 4.5) questions per session.

The Spanish corpora contains over 300 sessions (1,600 responses). The structure of questions and the technical environment are the same as for those of the English corpora—the speech recordings were done using computers within the ˜5 minutes time budget allocated per session. The average response length, measured in the number of words, is slightly less compared to its English counterpart Table 1.

During each session, the user completed a PHQ-8 (PHQ-9 after the suicidality question was removed). Since sessions are only a few minutes long, it was assumed users do not change state within them. Subsequently, PHQ-8 scores were mapped for binary classification. Scores at or above 10 mapped to presence of the condition; scores below 10 were mapped to absence of the condition.

| | | C | | E |
|---|---|---|---|---|
| | | English | D | Spanish |
| A | B | Train | Eval | Eval |
| Responses+ | | 16277 | 3139 | 292 |
| Responses− | | 41558 | 11395 | 1349 |
| Sessions | | 12872 | 3078 | 324 |
| Responses | | 179 | 162 | 110 |
| length (words) | | | | |
| Priors | | 0.281 | 0.216 | 0.178 |

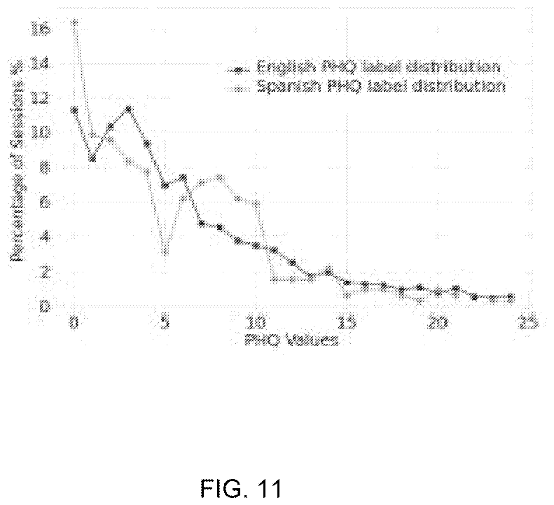

As described before, the data labels represent PHQ-8 questionnaire results. FIG. 11 depicts labels distribution for both English and Spanish test sets. As the data acquisition method as well as the demographics are similar it would be expected that the labels are as well. It is the case, for most PHQ scores there is an alignment between two data sets. There is a difference though, on the prevalence of these sets. The evaluation set is English and Spanish is 21% and 18% respectively. The largest outlier is the train set where there is the prevalence of 28%.

### Model Training

As stated in the data section, each interview with a patient is combined from multiple responses. Most of the NLP models (transformers) are initially built for the sequence of 512 input tokens. For the limited time and resource reasons no long sequence models were trained (e.g. Longfomer at the session level). As a result, if a session contains 5 responses, each response is treated as an independent record. All five responses had been attached to the same PHQ session label.

The study compares two different approaches. One is based on translating the Spanish test data to English using a translation service (e.g., a cloud-based translation service). For this purpose, we use English version of the ALBERT transformer language model. Its main advantage is the number of parameters that is substantially smaller than in most of other transformers (e.g. robustly optimized bidirectional encoder representations (RoBERTa)) and it is still effective in terms of performance than its counterparts. This approach may be used without any further training or tuning the original model if translation models between two languages are available. The classification training was done on only English data while the evaluation was conducted on English as well as translated Spanish into English sets. The training stage was done on small GPUs, 3×12 GB, for this reason the batch size was 9, learning rate 1e-6 and the training took 5 epochs.

Another approach is based on a pretrained multilingual model. This experiment uses a pretrained encoder/decoder MT5 model, trained on about 100 different language sets including English and Spanish among others. As NLP language models, in terms of parameters size, have grown to substantial volumes, due to limited hardware resources the disclosed approach does not compare like-for-like models (English vs Multilingual) in terms of number of parameters which is a common practice in the NLP space. The experiment uses a mT5 3 billion parameter model as its T5 counterpart GLUE benchmark is very similar to the ALBERT one. This allows to minimize the misinterpretation of the final performance results by trying to use the models that perform similarly on English language tasks. The mT5 training was done by utilizing only pre pre-trained encoder, that was used as a feature extractor in front of a classifier. The approach assumes that the output of the encoder is language agnostic and can be successfully used for multilingual classification purposes. A single supervised training approach without any language model finetuning has been applied here as well. The mT5 was trained on 65,000 English responses and evaluation was done on English development and test sets as well as Spanish test data. For the mT5 model, the main training characteristics were as follows: 1 epoch, 1e-5, batch size 25.

### Results

Mental health data sets are usually very imbalanced, the disclosed experiment uses the receiver operating characteristic area under the curve (ROC AUC) measure for measuring models performance of classification tasks. In addition, the disclosed experiment supplements the results with specificity and sensitivity equal error rate values. The disclosure proves that it is possible to build a different language depression screening model trained on English corpora. The ALBERT as well as mT5 model deliver comparable results to its English test counterpart. Both dev and test English sets return very similar results ˜0.73 ROC AUC. It is important to note that such results are expected based on the assumption that their BLEU benchmarks are also comparable. For the Spanish test set, the ALBERT architecture was slightly more effective than the MT5 at response level but after the data is aggregated mT5 significantly outperforms the English one. Aggregation to session level is a mean of response level predictions. In addition, the correlation coefficient of predictions at session level is 0.91.

| | | English Model | | | Multi Language Model | | |
|---|---|---|---|---|---|---|---|
| | Prevalence | roc_auc | specificity | Sensitivity | roc_auc | specificity | sensitivity |
| Responses | | | | | | | |
| En dev | 0.209 | 0.7437 | 0.6720 | 0.6722 | 0.7359 | 0.6718 | 0.6720 |
| En Test | 0.223 | 0.7380 | 0.6661 | 0.6658 | 0.7324 | 0.6637 | 0.6635 |
| Sp Test | 0.178 | 0.7486 | 0.6842 | 0.6849 | 0.7417 | 0.6716 | 0.6712 |
| Sessions | | | | | | | |
| En Dev | 0.206 | 0.8302 | 0.7528 | 0.7515 | 0.8372 | 0.7568 | 0.7569 |
| En Test | 0.218 | 0.8186 | 0.737 | 0.737 | 0.8160 | 0.7294 | 0.7308 |
| Sp Test | 0.182 | 0.7779 | 0.6981 | 0.6949 | 0.8004 | 0.6981 | 0.6949 |

When binary predictions are compared of both models against the true labels the similarity overlap is −92% (233+14+27+24). The largest difference is when ALBERT generates more false positive predictions (14 sessions) compared to four by mT5. When we artificially replace MT5 predictions with the ALBERT ones when the mT5 predicts 0 and ALBERT 1 then we see the 0.005 ROC AUC improvement across all three test and dev subsets. It indicates that through more sophisticated fusion methods it will be possible to further improve the performance on the evaluation sets. As a note, the internal best performance on our English test set is above 0.84 ROC AUC.

| True Labels | mT5 | ALBERT | Counts |
|---|---|---|---|
| 0 | 0 | 0 | 233 |
| 0 | 0 | 1 | 14 |
| 0 | 1 | 0 | 4 |
| 0 | 1 | 1 | 14 |
| 1 | 0 | 0 | 27 |
| 1 | 0 | 1 | 8 |
| 1 | 1 | 1 | 24 |

### DISCUSSION AND CONCLUSION

It is possible to predict depression in a different language (Spanish), in at least two ways, when the NLP model is trained using English data corpus. The model performance is effective using both methods. There is a 0.92 correlation between two approaches proposed in this disclosure where at the response level both models perform equally the same (−0.74 ROC AUC) but at the session level the multilingual one stands out.

1.-30. (canceled)

31. A method for detecting a behavioral or mental health condition, the method comprising:
(a) receiving an input signal comprising a plurality of audio or lexical characteristics of speech of a subject, wherein at least one of the plurality of audio or lexical characteristics of the speech relates to at least one language;
(b) based at least in part on the plurality of audio or lexical characteristics of the input signal, selecting one or more acoustic or natural language processing (NLP) models, wherein selecting one or more acoustic or NLP models comprises at least one of:
selecting a multilingual model,
selecting a model with a model language that includes the at least one language, selecting a model with a model language and translating the input signal from the at least one language to the model language, and
selecting a model with a model language and translating the model from the model language to the at least one language; and
(c) detecting a result estimating a severity of risk of having the behavioral or mental health condition by processing the input signal with a fused model or joint model derived from the one or more acoustic or NLP models.

32. The method of claim 31, wherein the input signal further comprises a plurality of lexical characteristics of speech of an agent.

33. The method of claim 31, further comprising:
detecting the at least one language; and
based on the at least one language:
selecting a model with a model language that includes the at least one language when the at least one language is from a database of supported languages; or
selecting a model with a model language that does not include the at least one language when the at least one language is not from a database of supported languages.

34. The method of claim 31, wherein the input signal comprises text or audio.

35. The method of claim 31, wherein selecting one or more acoustic or NLP models comprises selecting a model with a model language or dialect that is similar to the at least one language.

36. The method of claim 31, wherein processing the input signal with a single or fused model derived from the one or more acoustic or NLP models comprises (i) applying one or more weights to outputs of the one or more acoustic or NLP models; and (ii) combining the one or more weighted outputs.

37. The method of claim 36, wherein combining the one or more weighted acoustic or NLP models comprises using a machine learning model to combine the outputs of one or more acoustic or NLP models.

38. The method of claim 31, wherein the selecting the one or more acoustic or NLP models is also based at least in part on a latency of a prediction, a context of the input signal, or demographic information about the subject or the joint model or fused model derived from the at least one or more acoustic or NLP models further comprises at least one demographic or metadata-based model.

39. The method of claim 31, wherein the one or more NLP models include one or more of language-specific models, model-translation-based models, input signal-translation-based models, or multilingual models.

40. The method of claim 31, wherein:
prior to (b), determining one or more certainty measures, wherein a certainty measure of the one or more certainty measures corresponds to a language of the input signal, and determining at least one confidence measure based on at least one of the one or more certainty measures, wherein a confidence measure is associated with a performance, with respect to the language of the at least one certainty measure, of an acoustic model or NLP model of a plurality of acoustic or NLP models for detecting the severity of risk of having the behavioral or mental health condition;
in (b), selecting one or more acoustic or NLP models is based at least in part on the at least one confidence measure.

41. The method of claim 31, further comprising:
(d) based on a reliability measure of the result, iteratively or dynamically selecting one or more remedial actions and repeating (b) and (c) until a reliability threshold is achieved.

42. The method of claim 41, further comprising:
(e) based at least in part on the result, dynamically determining one or more remedial actions to the subject or to an agent to improve collection of the input signal.

43. The method of claim 40, wherein the certainty measure is generated at least in part using metadata of the input signal.

44. The method of claim 40, wherein the at least one confidence measure is based at least in part on a length of a session in which the input signal is recorded, a topic of the input signal, or a quality of the input signal.

45. The method of claim 31, wherein selecting one or more acoustic or NLP models comprises using the plurality of audio or lexical characteristics of the input signal to determine a weighted combination of the one or more acoustic or NLP models.
